(19) United States
(12) Patent Application Publication
(10) Pub. No.: US 2023/0390103 A1
(43) Pub. Date: Dec. 7, 2023

(54) MANDIBULAR REPOSITIONING DEVICE WITH LINGUAL FLANGE

(71) Applicant: Sleep Solutions of Texas, LLC, Tyler, TX (US)

(72) Inventor: Raghavendra Vitthalrao GHUGE, Tyler, TX (US)

(21) Appl. No.: 18/329,879

(22) Filed: Jun. 6, 2023

Related U.S. Application Data
(60) Provisional application No. 63/350,006, filed on Jun. 7, 2022.

(51) Int. Cl. A61F 5/56

(52) U.S. Cl. CPC A61F 5/566

(57) ABSTRACT

Mandibular apparatus have a mandibular tooth covering having a lingual flange extending caudally from a medial surface or posterior surface thereof. The lingual flange has a fin-shaped body that curves posteriorly as it extends caudally and is configured to position an apex thereof more posterior than a molar tooth of a user and that has a medial surface having a shape that is contoured to seat under the user's tongue in contact with the tongue, hyoglossus muscle, and genioglossus muscle, thereby simultaneously lifting the tongue and antero-cranially advancing the hyoid bone. A method of increasing the smallest concentric area of a user's upper airway using such mandibular apparatus are also included.

### TECHNICAL FIELD

The present disclosure relates generally to mandibular advancement devices, and more particularly to those having dual lingual flanges which are each a fin-shaped body curving posteriorly as it extends caudally and having a medial surface contoured to seat under a user's tongue in contact with the tongue, hyoglossus muscle, and genioglossus muscle, thereby simultaneously lifting the tongue and antero-cranially advancing the hyoid bone.

### BACKGROUND

The normal cross-sectional area of the narrowest point in a human upper airway (smallest concentric area (SMCA)) appears to be 140-180 mm². It is estimated that 30% of adults were born with a much smaller SMCA. These people have a SMCA of about 50-80 mm², which causes an increase in airflow resistance while awake and near total or total obstruction of the upper airway during sleep when the jaw, tongue, and associated muscles relax. This can affect this percentage of the population during any stage of life and can result in a variety of medical problems, especially those that are broadly described as or associated with obstructive sleep apnea (OSA).

With reference to FIG. 1, the visible tongue 2 appears to be a flat pear-shaped organ with an anterior apex and a posterior base. The tongue forms the oropharynx which is the superior limit of the collapsible upper airway. Underneath that surface the supporting muscles fan out in a primarily concave fan shape, both, antero-posteriorly and cranio-caudally. These muscles insert into the hyoid bone 4, which forms the caudal limit of the collapsible upper airway. The anterior and lateral walls of the upper airway are formed by the posterior elongated cranio-caudal limits of the hyoglossus muscle 6 and genioglossus muscle 8 complex of the tongue. They begin cranially from the ventral aspect of the visible surface of the tongue and descend caudally and posteriorly to insert into the superior, lateral, and anterior surface of the hyoid bone forming a little bit over 50% of the circumference of the collapsible upper airway. Relaxation of these muscles shrinks the size of the airway (transverse and anterior-posterior radii), displaces the hyoid bone 4 caudally, elongates the collapsible upper airway tube and simultaneously displaces the hyoid bone 4 posteriorly, thus narrowing almost the entire cross-sectional area of the collapsible upper airway along its entire length.

The body of the anterior vertical wall or aspect of the ventral aspect of the tongue is primarily made up by the genioglossus muscle 8 and the bulk of the posterior ventral aspect is made up by the hyoglossus muscle 6. The genioglossus muscle 8 forms a fan shape structure from center bilaterally and forms a fan shape cranial to caudal as well. It originates from the ventral surface of the tongue, fans out and descends caudally curvilinearly bilaterally and finally inserts caudally into the superior-posterior surface of the anterior aspect of the hyoid bone 4. The hyoglossus muscle 6 forms a vertical curvilinear fan shape as it descends caudally and posteriorly to attach to the body 30, horns 32 (lesser cornu), and greater cornuae 34 of the hyoid bone 4. Relaxation of these muscle results in posterior and caudal displacement of both the hyoid bone 4 and the vertical cranio-caudal anterior wall of the upper airway.

Incumbent and all prior oral appliances, by their very design, are designed to anteriorly advance the mandible thus pulling the geniohyoideus 10 and horizontal fibers of the genioglossus 8 anteriorly. This tends to pull the anterior aspect of the hyoid bone 4 anteriorly. In extremes of mandibular advancements, these oral appliances may even tend to cantilever the greater and lesser cornu 34, 32 of the hyoid bone caudally (since they do not support the hyoglossus 6 simultaneously) and elongate the collapsible upper airway further defeating any advantage gained from the anterior displacement of the body 30 of the hyoid bone 4. These oral appliances are unable to cranially raise or support the hyoglossus 6 and they are unable to leverage the vertical and oblique component that forms the bulk of the genioglossus 8; thus, unable to simultaneously, caudally, and anteriorly, advance the hyoid bone 4 and the anterior wall of the collapsible upper airway supported by it. Thus, sleep-induced relaxation of the hyoglossus 6 persistently closes the airway, especially in severe cases of OSA, even while using existing oral appliances.

Medically, a continuous positive airway pressure (CPAP) device is prescribed for OSA treatment. The CPAP operates to fill the upper airway with compressed air; thus, preventing its collapse or narrowing and does so quite well when successfully used each and every night. Prior historical data reveals that 59% of CPAP's or BIPAP's are rejected by individuals with OSA each year due to intolerance to CPAP or failed therapy. This abandonment of treatment causes huge economic loss and health complications. More recently, the recall of six million CPAP devices worldwide due to potential carcinogenic risk has brought to the forefront a major risk of CPAP devices. It has dealt a major blow to the authenticity and trust in such machines.

Historically, mandibular advancement devices (MAD) or mandibular repositioning devices (MRD) can be used as another therapy option for OSA and severe OSA, especially after failed CPAP therapy. The limits of this therapy are discussed above. There is no single oral appliance (MAD or MRD) on the market that has FDA clearance for severe OSA. The vast majority of patients that suffer from severe OSA are left without a good sustainable non-invasive therapeutic option.

Surgeries like uvulo-palato-pharyngoplasty or laser assisted uvuloplasty, which gained popularity in the 90's and early part of this century, have fallen to the wayside due to failure in their ability to treat severe OSA. Even hyoid lift surgery is unable to control OSA by itself because it only addresses the hyoglossus without addressing the genioglossus muscle simultaneously.

More recently, very expensive ($40,000-$130,000) surgeries to implant a pacemaker for the tongue have shown dismal outcomes when electrification of the horizontal aspect of the genioglossus alone is attempted. This surgery technique has only penetrated 0.1% of the market. Other attempts at electrification of the tongue by using a transcutaneous electrical nerve stimulation during the day have met with small success in treating primary snoring (without OSA) but are completely ineffective for moderate and severe OSA.

Clearly, the medical and dental industries have failed to provide a sustainably effective treatment for moderate and severe OSA. Thus, there is a need for a non-invasive, lower cost, effective treatment for moderate and severe OSA.

### SUMMARY

In one aspect, mandibular apparatus have a mandibular tooth covering that includes a lingual flange extending caudally from a medial surface or posterior surface thereof. The lingual flange has a fin-shaped body that curves posteriorly as it extends caudally and is configured to position an apex thereof more posterior than a molar tooth of a user and that has a medial surface having a shape that is contoured to seat under the user's tongue in contact with the tongue, hyoglossus muscle, and genioglossus muscle, thereby simultaneously lifting the tongue and antero-cranially advancing the hyoid bone.

In one embodiment, the lingual flange extends from the medial surface proximate a front incisor portion of the mandibular tooth covering and bifurcates into a left lingual portion and right lingual portion that each terminate with the fin-shaped body.

In one embodiment, the mandibular device includes dual lingual flanges, one extending from the medial surface proximate a left molar portion and another extending from the medial surface proximate a right molar portion of the mandibular tooth covering.

In all embodiments, the fin-shaped body can be angled medially inward as it extends caudally, and the lateral surface of the fin-shaped body can be spaced a distance medially away from the molar portion of the mandibular tooth covering. The medial surface of the fin-shaped body is typically generally convex along the length thereof.

In one embodiment, the apex has a rounded tip. The rounded tip can be convex medially, laterally, anteriorly, and posteriorly.

In one embodiment, the mandibular tooth covering has a right lingual flange and a left lingual flange. From a top view, the apex of the right lingual flange is rotated counterclockwise relative to an outermost surface of the medial surface of the right lingual flange and the apex of the left lingual flange is rotated clockwise relative an outermost surface of the medial surface of the left lingual flange. The left and right lingual flanges can be mirror images of one another.

In any of the embodiments, the fin-shaped body can define an internal cavity having therein a sensor operatively seated therein in communication with the oral cavity to measure or sense a variable thereof. The fin-shaped body can include a muscle stimulator operatively seated within the internal cavity and configured for contact with the tongue, hyoglossus muscle, genioglossus muscle, or a combination thereof. Here, the mandibular apparatus has an electronic transmitter and an electronic receiver in operative electrical engagement with the sensor and/or the muscle stimulator, wherein the electronic transmitter and an electronic receiver are configured to communicate with an external controller for dynamic operation of the sensor and/or muscle stimulator.

In all embodiment, the mandibular apparatus may have a protrusive flange extending cranially from a postero-lateral surface of the mandibular tooth covering.

In another aspect, methods of increasing the smallest concentric area of a user's upper airway are disclosed. The methods includes identifying a user in need of an increased area for their smallest cross-sectional area of their upper airway, fitting any of the mandibular apparatus disclosed herein to the user's teeth, postero-ventral surface of the user's tongue, genioglossus muscle, and hyoglossus muscle, and providing the mandibular apparatus thus fitted to the user and instructing the user to wear the mandibular apparatus while awake, while exercising, and/or during sleep.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present system.

FIG. 1 is a representation of the tongue and the muscles thereunder.

FIG. 2 is a representation of the hyoid bone below the mandible.

FIG. 3 is a top, posterior perspective view of a first embodiment of a mandibular apparatus.

FIG. 4 is a posterior, perspective view of a second embodiment of a mandibular apparatus.

FIG. 5 is a posterior view of right portion of a third embodiment of a mandibular apparatus.

FIG. 6 is a left side view of the first embodiment of FIG. 3.

FIG. 7 is a top view of a fourth embodiment of a mandibular apparatus.

FIG. 8 is an enlarged view of the medial surface of the right, hollow lingual flange of FIG. 3 using dashed boxes to represent electronic features present inside the internal cavity thereof.

FIG. 9 is a top view of a fifth embodiment of a mandibular apparatus.

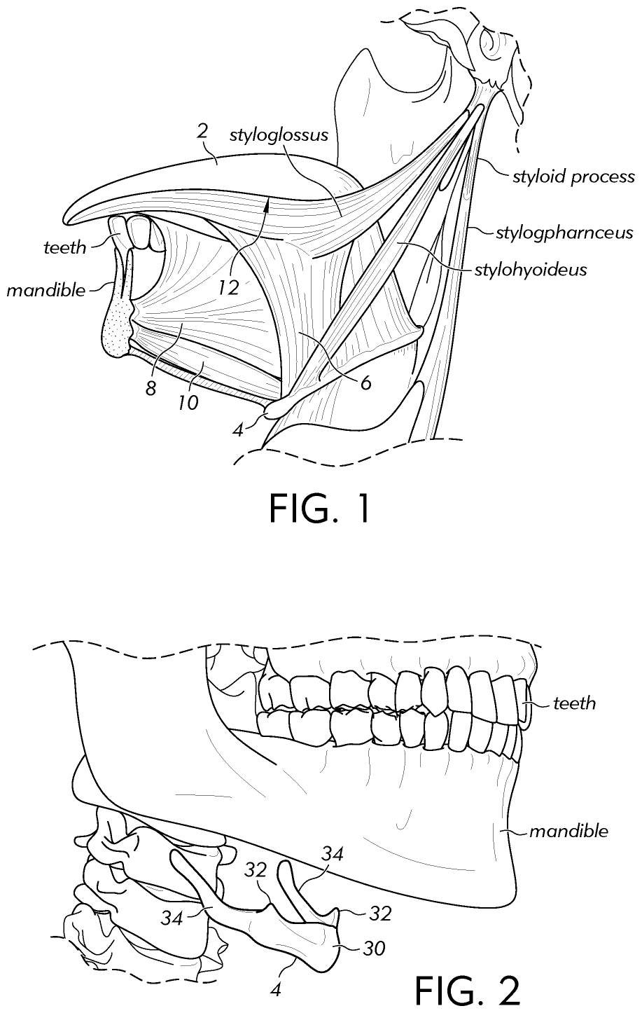

### DETAILED DESCRIPTION

The following detailed description will illustrate the general principles of the invention, examples of which are additionally illustrated in the accompanying drawings. In the drawings, like reference numbers indicate identical or functionally similar elements.

“About” as defined herein with respect to lineal measurement is defined as +/−0.3 mm, with respect to angles is defined as +/−3°, more preferably +/−2°, and even more preferably +/−10.

Referring to all embodiments of mandibular advancement devices (MADs) disclosed herein, each has a mandibular tooth covering 102 having a lingual flange 104 extending caudally from a medial surface 110 or a posterior surface 111 thereof. The lingual flange 104 has a fin-shaped body 106 that curves posteriorly as it extends caudally to position an apex 108 thereof more posterior than the posterior surface 111 of the mandibular tooth covering 102. The apex 108 is a rounded tip. The rounded tip is is convex medially, laterally, anteriorly, and posteriorly. The fin-shaped body 106 has a medial surface 120 having a shape that is contoured to seat under a user's tongue in contact with the tongue, hyoglossus muscle, and genioglossus muscle and is configured to simultaneously lift the tongue and antero-cranially advance the hyoid bone. All embodiments herein provide antero-cranial stabilization of the genioglossus-hyoglossus muscle complex creating an antero-cranial stabilization, lift and advancement of the tongue and hyoid bone while being worn, whether during sleep or while awake, which increases the SMCA and decreases the vertical length of the upper airway, simultaneously, thereby reliably and consistently increasing airflow volume, decreasing airflow resistance, and keeping the airway wide open. The end result is increased oxygen delivery to the body while worn by a user.

The fin-shaped body 106 of each lingual flange is configured to sit underneath the user's tongue while avoiding the midline structures of the floor of the mouth, whereby the weight of the tongue secures the lingual flanges in place. Moreover, the pressure of the tongue keeps the mandibular advancement apparatus in contact with the postero-ventral surface of the user's tongue to mechanically provide stabilization of the tongue in an antero-cranial position and to further elevate the tongue and advance the hyoid bone antero-cranially. This contact provides an increase in muscle tone through direct activation of the touch receptors of said surfaces, thus creating a natural anterior advancement of the tongue during wake and sleep, which ultimately is effective without the mandibular advancement device because of the increased muscle tone.

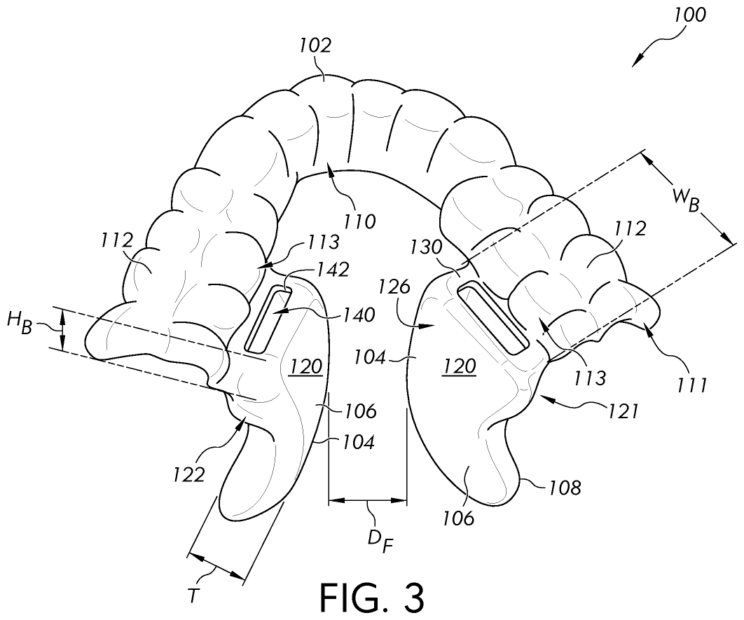

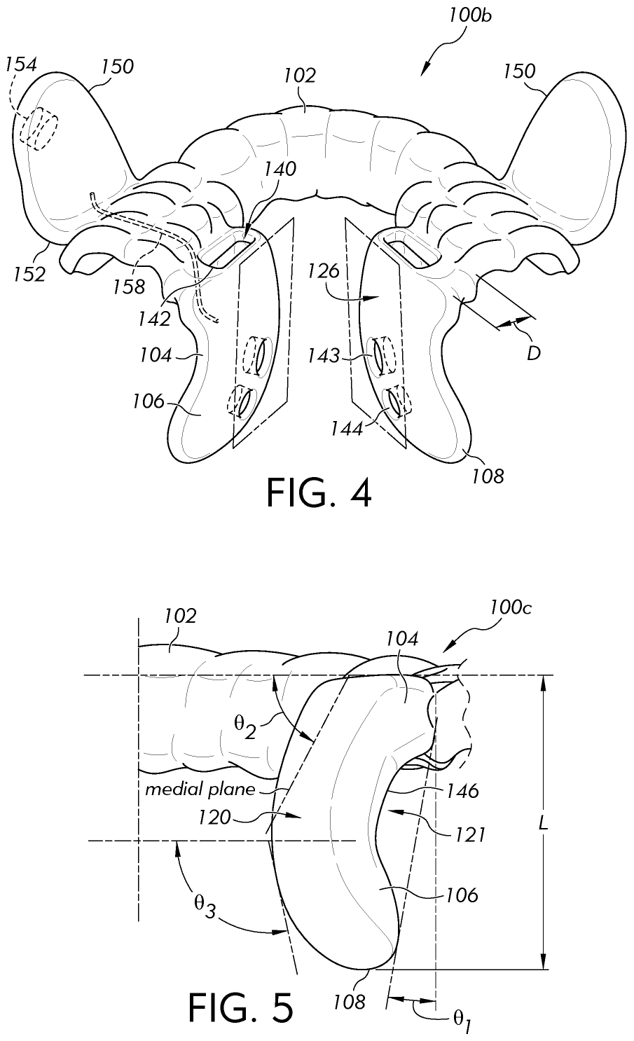

The features of the fin-shaped body will be described with respect to the embodiment of FIGS. 3 and 4, but is applicable to all embodiments. Here, the mandibular advancement apparatus 100 has a left and a right lingual flange 104 respectively extending caudally from a left and a right postero-medial surface 113 proximate a respective molar portion 112 of the mandibular tooth covering 102. The molar portion 112 can comprise the last two or three mandibular molar (tricuspid) teeth on the right and left side alike. Typically, the left and right lingual flanges 104 are mirror images of one another, i.e., identical in size and shape, but is not limited thereto. They can be configured to be non-identical to adapt to the shape of a user's tongue and muscle structure if it is not bilaterally symmetrical.

With reference to FIGS. 4 and 5, the fin-shaped body 106 is angled medially inward as it extends caudally (θ₁) and the lateral surface 121 of the fin-shaped body is spaced a distance (D) medially away from the molar portion of the mandibular tooth covering. From the rear perspective view, as shown in FIG. 4, the apex 108 of the right lingual flange is rotated counterclockwise relative to an outermost surface of the medial surface 120 of the right lingual flange and the apex 108 of the left lingual flange is rotated clockwise relative an outermost surface of the medial surface 120 of the left lingual flange.

The medial surface 120 of the fin-shaped body 106 is generally convex along the length thereof. The posterior surface 122 is generally convex cranially and transitions to concave caudally therefrom until it convexly forms the apex 108, thereby forming a convex-concave-convex surface from cradial to caudal. These two surfaces are configured for direct, continual contact with the ventral postero-caudally convex-concavely spreading fan-shaped surface of the tongue including the genioglossus muscle and the hyoglossus muscle along its entire course as the tongue descends caudally and posteriorly towards the hyoid bone. In another embodiment, the medial surface 120 can be concave or concavo-convex, or linear so long as it is configured to remain in contact with the ventral surface of the tongue as it descends postero-caudally and laterally towards the hyoid bone. A snug continuous fit is desired with the ventral surface of the tongue.

Turning now to FIG. 5, the medial plane represents the cranial medial surface of the fin-shaped body 106 of the lingual flange 104. The angle between this plane and the Y-axis (runs horizontally through the left and right cranial surfaces of the mandibular piece) in a centric occlusion position (mouth closed), from an in antero-posterior view, is a medial angle. The medial angle (θ₂) is measured medially and is an acute angle in a range of about 62° to about 88°, more preferably about 70° to about 72°. The lateral plane angle (θ₃) is the angle between the medial surface of the fin-shaped body and the transverse mandibular plane in a dorsal view or cranio-caudal view (Z-axis) of the mandibular piece 102. θ₃ is measured medially and is an obtuse angle in a range of about 980 to about 112°, more preferably about 103° to about 108.6°.

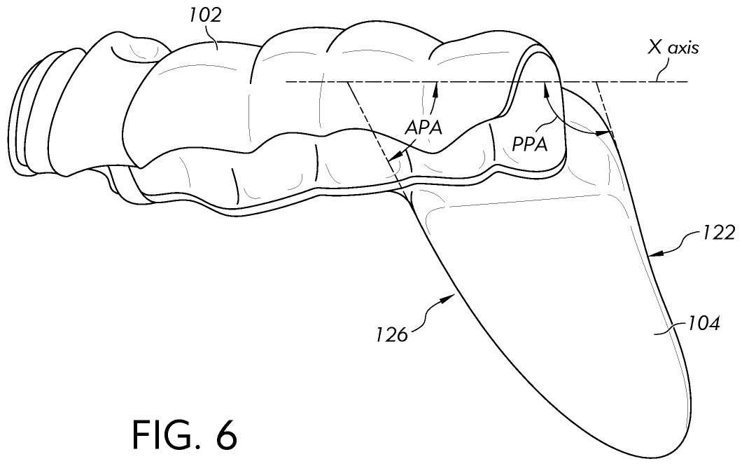

As shown in FIGS. 3 and 4, the fin-shaped bodies 106 are hollow, thereby defining an internal cavity 140 in which a sensor may be operatively positioned, and have an opening 142 providing access to the internal cavity 140 and one or more apertures 143, 144 for electronic components that need access to the external environment. The sensor, not shown, can have a sensing end positioned in one or both of the apertures 143, 144 present in the medial surface 120 thereof (see FIG. 8). The apertures 143, 144 may alternately be in another surface of the flange 104. The apertures 143, 144 may each comprise a sensor operatively seated therein to communication with the user's mouth and/or for terminals of a power charging system to charge a rechargeable battery stored in the internal cavity 140. However, in other embodiments, such as FIG. 6, one or both fin-shaped bodies can be solid.

Turning again to FIG. 3, a base 130 of each lingual flange forms the attachment junction to the tooth covering 102. The base 130 may be about 10 mm to about 12 mm wide (W_{B}) and have a height (H_{B}) of about 4 mm to about 7 mm. The base 130 can be wedged-shaped, in that it has a greater thickness caudally than it does cranially, which contributes to the angle (θ₁) of the fin-shaped body in the medial direction, shown in FIG. 6. The cranial thickness of the wedge-shaped base 130 may be in a range of about 2 mm to about 4 mm and the caudal thickness may be in a range of about 4 mm to about 6 mm. The base 130 can be rigid, soft, or flexible. As the fin-shaped body 106 extends from the base 130, it gradually tapers to a narrower width as it descends caudally. The thickness is about 10 mm to about 12 mm at the base and tapers to about 7 mm to about 10 mm then to about 4 mm to about 6 mm before or at the turn posteriorly to form the apex, which has a thickness of about 3 mm to about 4 mm. The length (L) of the fin-shaped body, see FIG. 5, is in a range of about 14 mm to about 18 mm. For the average user, the length is usually about 16 mm.

In all aspects, the fin-shaped body can have overall dimension in the cranio-caudal direction (length) in a range of about 18 mm to about 30 mm, more preferably about 23 mm to about 24 mm; in the antero-posterior direction (width) in a range of about 8 mm to about 20 mm, more preferably about 12 mm to about 15 mm; and in the medio-lateral direction (thickness) in a range of about 2 mm to about 10 mm, more preferably about 4 mm to about 6 mm. The dimensions described herein are exemplary and are not meant to restrict the construction of the MAD.

As best seen in FIG. 5, the lateral surface 121 is concave or has an indentation 146 below the base 130 of the lingual flange 104. The concave or indented shape of the lateral surface 121 is configured to create a recess to accommodate the medial bulging or deformities of the gingival tissue without rubbing thereagainst, i.e., providing clearance. In other embodiments, the lateral surface 121, may be concave or convex or linear and flat vertically and transversely as needed to be configured to accommodate dental and gingival variations of a user.

Referring again to FIGS. 3 and 4, the anterior surface 126 is convex in shape throughout the entire cranio-caudal course of the fin-shaped body 106. The fin-shaped body 106 may be configured to be flexible to allow movement with the tongue or may be rigid. A rigid fin-shaped body provides mechanical support and leverage and sensory functionality. An elastic fin-shaped body provides sensory and electrode functionality and pharmacotherapeutic functionality while it enlarges the cross-sectional area and decreases the linear dimension of the airway.

The apex 108 is a pyramidal structure or a conical structure that is wider in antero-posterior dimension than in its medio-lateral dimension. The convex curvature increases at the tip as it then smoothly forms a caudal smooth bluntness at the tip. Essentially, all surfaces forming the apex have terminal convexities and come together as a smooth rounded tip that is convex in all directions. The apex 108 also undergoes torsion around the long axis of the fin-shaped body and along the anterior and posterior surfaces. This torsion is configured to angulate the apex 108 away from the concave fanning out of the postero-caudal surfaces of the tongue laterally on each side, both, to the left and right as represented in FIG. 4 and described above. Thus, viewed from the cranial surface, the right apex is rotated counterclockwise while the left apex is rotated clockwise. This may be reversed in another embodiment where the medial surfaces of the lingual flanges are concave and oriented facing medially at the apex. The apex 108 may be solid or hollow. The apex 108 can be flexible for movement with the tongue or it can be rigid. An aperture for an electrode or other muscle stimulator can be positioned in any surface of the fin-shaped body that contacts a muscle, including the apex.

Turning now to FIG. 6, either the anterior plane or the posterior plane is used to determine the X-axis orientation of the lingual flange. The X-axis is a horizontal antero-posterior plane of the mandibular piece 102 configured when the mouth is closed, i.e., in a centric occlusion position. The anterior plane is defined as the plane of the anterior surface of the lingual flange 104 (measured at the highest point of the body of the lingual flange) that transverses the X-axis with the mouth in the centric occlusion position, thereby defining the anterior plane angle (APA). The APA is typically within a range of about 450 to about 90°, more preferrably about 53.7° to about 60.8°. An alternative to measuring the APA is the posterior plane angle (PPA). This is the anterior angle between the posterior plane of the lingual flange and the X-axis. The PPA is typically within a range of about 90° to about 135°, more preferably about 113.7° to about 123.6°. Only one of the two angles (APA or PPA) is used at any given time for determining the X-axis orientation.

The axes and angles discussed herein are used to position the lingual flange 104 such that the superior aspect of the base 130 aligns with the existing superior surface of the molar portion 112 of the mandibular piece covering 102 and to provide vertical lift and antero-cranial advancement of the hyoglossus and genioglossus muscle complex while the mouth is in the centric occlusion position and as the mandible experiences caudal rotation. The lingual flange extends about 18 mm to about 22 mm caudally below the inferior (caudal) most edge of the mandibular tooth covering 102 as fitted to a specific user's oral cavity, tongue, and mandible. The right and left lingual flanges 104 are configured such that the minimum distance between the two (D_{F}) is in a range of of about 7.88 mm to about 22 mm, more preferably about 8.64 mm to about 15 mm. This distance may be configured so that the two lingual flanges are placed one on either side of the ventral lingual frenulum for ease of use and a comfortable snugness with the ventral surface of the tongue. This configuration provides the best anterior and cranial advancement of the entire anterior wall of the upper airway including its most caudal limit, the hyoid bone itself.

The mandibular advancement apparatus disclosed herein can be made by any known or hereinafter developed methods using materials compatible with human intra-oral use for prolonged periods of time. The lingual flange 104 may be manufactured together with the oral appliance (tooth covering 102) as one piece or attached to the oral appliance through one of the many known methods or hereinafter developed methods. The fundamental description of the lingual flange 104 herein provides adequate description for one of skill in the art of making oral appliances to make the apparatus disclosed herein.

Referring now to the embodiment of FIG. 4, the mandibular apparatus 100b includes a left protrusive flange 150 and a right protrusive flange 150 as described in pending U.S. application Ser. No. 17/098,355, filed Nov. 14, 2020 as well as the left and right lingual flanges 104 described above. The entire contents of the '355 application are incorporated herein by reference. The left and right protrusive flanges 150 each extend cranially from a postero-lateral surface of the mandibular tooth covering 102. The protrusive flanges 150 may be hollow, thereby defining an interior cavity, and have an opening 152 providing access to the internal cavity. The lateral surface of either or both of the protrusive flanges 150 can have a through port 154 into the internal cavity. The through port 154 may have a sensor head of a sensor housed in the internal cavity or an electrode of a power charging device or any other electronic features and/or deviced described in the '355 application.

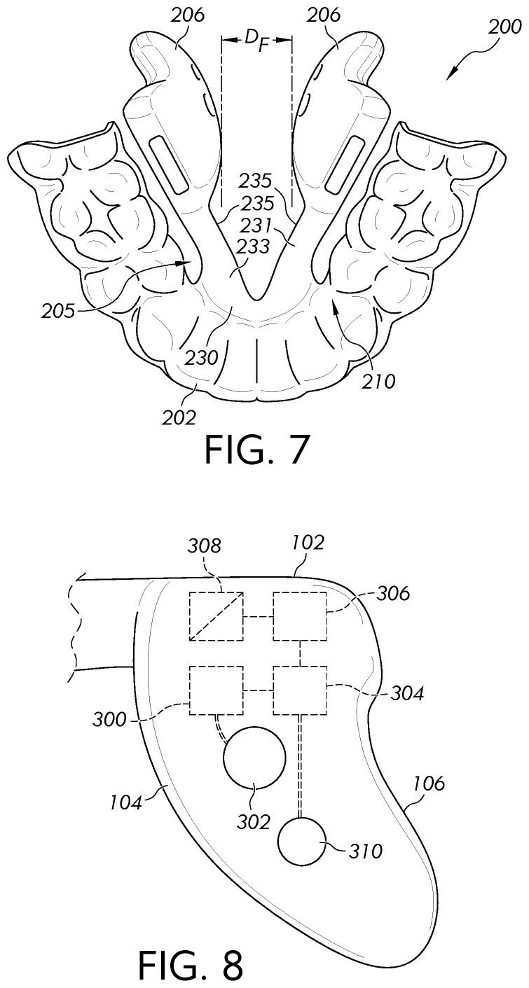

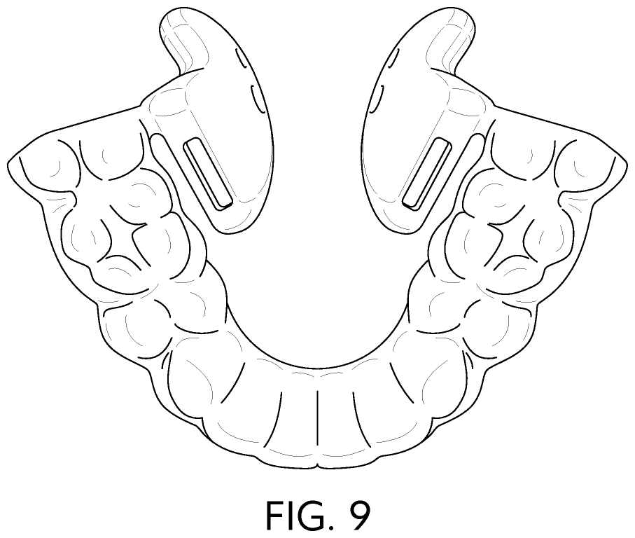

While FIGS. 3-6 and 9 show embodiments with left and right lingual flanges 104 extending for the left and right molar portions 112, respectively, the mandibular advancement apparatus 200 of FIG. 7 is constructed with a single flange 205 that extends posteriorly from the medial surface 110 of a mandibular tooth covering 202 proximate the front incisor portion 213 and bifurcates into a left lingual portion 231 and right lingual portion 233 as shown in FIG. 7, which each have an arm 235 terminating with a fin-shaped body 206 as described above for the dual lingual flange embodiments. The fin-shaped bodies 206 can have shapes, dimensions, and characteristics that are the same as described above, whether hollow or solid and/or flexible or rigid. The minimum distance (D_{F}) between the two portions 231, 233 is selected such that the tongue cannot slip through and get underneath the base 230, which is the attachment point to the medial surface 210 of the mandibular tooth covering 202, or arms 235. Acceptable ranges are presented above for D_{F}.

Referring now to FIG. 8, in all embodiment having one or more hollow lingual flanges 106, 206, a power source 300 is positioned in the internal cavity of the lingual flange and is electrically connected for electrical communication with all other systems and electronics housed in the internal cavity thereof. The power source 300 may be a rechargeable battery that is operatively, electrically connected to a charging terminal 302 that is seatable in mated engagement with a charging system when not in a user's mouth. In the embodiment of FIG. 4, the power source may be in the internal cavity of the protrusive flange 150 and an electrical connection (such as a wire 158) can extend from this power source to the electrical components in the lingual flange. Alternately, the power source 300 may be in the internal cavity 140 of one or both of the lingual flanges 104 and an electrical connection can extend from this power source to the electrical components in the protrusive flange 150 to power its electrical components.

Returning to FIG. 8, non-transitory memory 304 can be present in the internal cavity in operative electrical communication with the power source 300, a computer processing unit 306 (or it can be part of the CPU), a transmitter and a receiver 308, and an oral cavity active element 310. The oral activity active element 310 may be a sensor, an electrode, or other electronics. If it is a sensor it may a pulse oxygen sensor, a vibration and airflow sensor, a pH sensor, a doppler ultrasound sensor, an M-Mode ultrasound sensor, a 2D ultrasound sensor, 3D ultrasound sensor, a pressure plate sensor for measuring bruxism, a pulse transit time sensor, electrocardiography (EKG), electroencephalography (EEG), electromyography (EMG), electrooculography (EOG), lactic acid sensor, hygroscopic/hydration sensor, video and audio recording, non-invasive systolic/diastolic blood pressure sensor, a carotid doppler (trans-oral) sensor, and a cardiac trans-oral echocardiography sensor, thermometer, blood glucose sensor. If it is an electrode, it may stimulate the muscles of and/or under the tongue, speech, and/or swallowing. Other electronic components may be included in the internal cavity in operative electronic communication with any of the other components as needed. These electronics can be configured for use with a controller and the systems described in the '355 application.

### Working Example

A CAD (computer-aided design) program was used to design the dimensions and shape of a lingual flange. The lingual flange is attached to the mandibular piece in the CAD program to satisfy the X, Y and Z-axes of the product. The completed design is then printed using a stereolithography (SLA) 3D printer to print the mandibular piece with a left lingual flange and a right lingual flange fused thereto using a bio-compatible resin. The 3D printed piece is then subjected to post-processing curing and finished appropriately according to the manufacturer's specification for the bio-compatible resin. SLA printers are available from Form Labs, Somerville, Massachusetts or from BencoDental, Texas.

The lingual flanges were set to have a convex medial surface, APA=54°, θ₂=70° and θ₃=103°, a total length=23 mm, a maximum W_{B} dimension of 13 mm, a maximum thickness (T) of 6 mm, and a minimum D_{F}=8.64 mm. This configuration provides significant cranial elevation of the entire ventral surface of the tongue, anterior displacement of the entire vertical anterior wall of the upper airway and elevates the hyoid bone along with the genioglossus muscle and hyoglossus muscle even while awake. The mandibular covering is configured to rotate and descend caudally with mouth opening. This caudal rotation leverages the lingual flanges and rotates the lingual flanges cranially and anteriorly (like a see-saw). The see-saw antero-cranial displacement of the lingual flanges simultaneously displaces these supported muscle groups antero-cranially, thereby antero-cranially displacing the entire anterior upper airway wall and the hyoid bone.

Here, one degree of caudal rotation of the mandible (configured to represent the same amount of mouth opening) will, at the very least, elevate the posterior surface of the lingual flange at the tip of the apex 108 by one degree also, approximately 0.4 mm of antero-cranial displacement along an arc. The calculation is for every 1° of arc, 10° (2*π*23 mm)/360°. Since the medial and posterior surfaces of the lingual flanges are typically configured to caudally support and remain in constant contact with the ventral surface of the tongue, all these structures are also predicably elevated antero-cranially by 0.4 mm along an arc for every degree of caudal rotation of the mandibular piece. Thus, a 25 mm caudal rotation of the mandibular piece would antero-cranially displace the apex of the lingual flange by 10.04 mm. This lift would simultaneously antero-cranially lift the attached hyoid bone by a similar amount while awake and a slightly lesser amount during sleep (accounting for muscle relaxation), thus resulting in an increase in the radius and cross-sectional area of the collapsible upper airway along its entire vertical length while decreasing the total length of the airway through antero-cranial lift of the hyoid bone that forms the very caudal limit of the upper airway.

While the inventor's recent oral appliances have focused on antero-cranial advancement of the mandible to open the upper airway when the jaw relaxes during sleep or the mouth is opened while awake, in particular to treat sleep apnea. Since the genioglossus muscle and hyoglossus muscle become relaxed during sleep, these muscles may not entirely follow the antero-cranial advancement of the mandibular piece. Additional or alternate means to open the upper airway is advantageous, especially one that interacts with these muscles. The lingual flanges described herein form an elevated platform configured to overcome the midline obstacles (frenulum and ventral body of the tongue) and physically/mechanically support the ventral surface of tongue and the genioglossus-hyoglossus muscle complex from underneath, which increases the SMCA of the entire collapsible upper airway. The apparatus with such lingual flanges can be used to treat sleep apnea, even severe sleep apnea, as well as during physical activities while awake where increased oxygen intake is beneficial.

A mandibular advancement apparatus with lingual flange(s) as described herein have the advantage of counter-leverage provided by the lingual flanges when the mandibular piece is in a centric occlusion position (mouth closed), thereby preventing caudal rotation of the mandibular piece and preventing the mouth from opening (mouth opening occurs when the mandibular piece rotates caudally even when the mouth is meant to be closed). As such, dry mouth and dental damage is eliminated. Another advantage is that the lingual flange(s) reduce the amount of anterior advancement necessary; thus reducing stress on the temporo-mandibular joint (TMJ), which should reduce TMJ complications such as joint pain.

Each apparatus described herein is configured to provide the antero-cranial stabilization of the tongue when the mouth is closed, i.e., in a centric occlusion position. This prevents the sleep-related relaxing of the genioglossus-hyoglossus muscle complex which is in a postero-caudal direction. Each apparatus provides a non-surgical means for increasing the SMCA for the user and an alternative to a CPAP machine. Additionally, each apparatus is configured to lift the hyoid bone when in use, but returns and leaves the hyoid bone in its natural position when not in use.

Each apparatus is useful while awake (during physical activity) and while asleep. Each apparatus decreases breathing effort by eliminating the Bernoulli effect and increasing the tidal volume and gas exchange. These attributes have the potential to provide greater amounts of oxygen delivery as compared to natural breathing during physical activity or during sleep. Previous oral appliances for sleep apnea have historically not been used to improve oxygenation for physical activity during wake.

It should be noted that the embodiments are not limited in their application or use to the details of construction and arrangement of parts and steps illustrated in the drawings and description. Features of the illustrative embodiments, constructions, and variants may be implemented or incorporated in other embodiments, constructions, variants, and modifications, and may be practiced or carried out in various ways. Furthermore, unless otherwise indicated, the terms and expressions employed herein have been chosen for the purpose of describing the illustrative embodiments of the present invention for the convenience of the reader and are not for the purpose of limiting the invention. Having described the invention in detail and by reference to preferred embodiments thereof, it will be apparent that modifications and variations are possible without departing from the scope of the invention which is defined in the appended claims.

What is claimed is:

1. A mandibular apparatus comprising:
a mandibular tooth covering having a lingual flange extending caudally from a medial surface or posterior surface thereof;
wherein the lingual flange has a fin-shaped body that curves posteriorly as it extends caudally and is configured to position an apex thereof more posterior than a molar tooth of a user and that has a medial surface having a shape that is contoured to seat under a user's tongue in contact with the tongue, hyoglossus muscle, and genioglossus muscle, thereby simultaneously lifting the tongue and antero-cranially advancing the hyoid bone.

2. The mandibular apparatus of claim 1, wherein the lingual flange extends from the medial surface proximate a front incisor portion of the mandibular tooth covering and bifurcates into a left lingual portion and right lingual portion that each terminate with the fin-shaped body.

3. The mandibular apparatus of claim 1, comprising dual lingual flanges, one extending from the medial surface proximate a left molar portion and another extending from the medial surface proximate a right molar portion of the mandibular tooth covering.

4. The mandibular apparatus of claim 1, wherein the fin-shaped body is angled medially inward as it extends caudally.

5. The mandibular apparatus of claim 4, wherein the lateral surface of the fin-shaped body is spaced a distance medially away from the molar portion of the mandibular tooth covering.

6. The mandibular apparatus of claim 4, wherein the medial surface of the fin-shaped body is generally convex along the length thereof.

7. The mandibular apparatus of claim 1, wherein the apex is a rounded tip.

8. The mandibular apparatus of claim 7, wherein the rounded tip is convex medially, laterally, anteriorly, and posteriorly.

9. The mandibular apparatus of claim 1, wherein the mandibular tooth covering has a right lingual flange and a left lingual flange.

10. The mandibular apparatus of claim 9, wherein, from a top view, the apex of the right lingual flange is rotated counterclockwise relative to an outermost surface of the medial surface of the right lingual flange and the apex of the left lingual flange is rotated clockwise relative an outermost surface of the medial surface of the left lingual flange.

11. The mandibular apparatus of claim 9, wherein the left and right lingual flanges are mirror images of one another.

12. The mandibular apparatus of claim 1, wherein the fin-shaped body defines an internal cavity and (a) a sensor is operatively seated within the internal cavity, wherein the sensor communicates with the oral cavity to measure or sense a variable thereof and/or (b) a muscle stimulator is operatively seated within the internal cavity and is in contact with the tongue, hyoglossus muscle, genioglossus muscle, or a combination thereof.

13. The mandibular apparatus of claim 12, comprising an electronic transmitter and an electronic receiver in operative electrical engagement with the sensor and/or the muscle stimulator, wherein the electronic transmitter and an electronic receiver are configured to communicate with an external controller for dynamic operation of the sensor and/or muscle stimulator.

14. The mandibular apparatus of claim 1, further comprising a protrusive flange extending cranially from a postero-lateral surface of the mandibular tooth covering.

15. A method of increasing the smallest concentric area of a user's upper airway, the method comprising:
identifying a user in need of an increased area for their smallest cross-sectional area of their upper airway;
fitting a mandibular apparatus according to claim 1 to the user's teeth, postero-ventral surface of the user's tongue, genioglossus muscle, and hyoglossus muscle; and
providing the mandibular apparatus thus fitted to the user.

16. The method of claim 15, comprising instructing the user to wear the mandibular apparatus while awake.

17. The method of claim 16, comprising instructing the user to wear the mandibular apparatus during exercise.

18. The method of claim 15, comprising instructing the user to wear the mandibular apparatus during asleep.

19. The method of claim 15, wherein the lingual flange extends from the medial surface proximate a front incisor portion of the mandibular tooth covering and bifurcates into a left lingual portion and right lingual portion that each terminate with the fin-shaped body.

20. The method of claim 15, comprising dual lingual flanges, one extending from the medial surface proximate a left molar portion and another extending from the medial surface proximate a right molar portion of the mandibular tooth covering.
